(19) United States
(12) Patent Application Publication
(10) Pub. No.: US 2023/0088064 A1
(43) Pub. Date: Mar. 23, 2023

(54) EXTERNAL MARKING MATERIAL FOR MEDICAL IMAGING PROCEDURES

(71) Applicants: Francisco J. OSSE, Osasco (BR); Ghassan S. KASSAB, La Jolla, CA (US)

(72) Inventors: Francisco j. Osse, Sao Paolo (BR); Ghassan S. Kassab, La Jolla, CA (US)

(21) Appl. No.: 17/909,229

(22) PCT Filed: May 3, 2021
(86) PCT No.: PCT/US2021/030522

§ 371 Date: Sep. 2, 2022

Related U.S. Application Data
(60) Provisional application No. 63/100,222, filed on Mar. 3, 2020.

(51) Int. Cl. A61B 6/04

(52) U.S. Cl. CPC A61B 6/0492

(57) ABSTRACT

The invention comprises a substance suitable for external marking of a patient's skin during a radiographic procedure, such as angiography and endovascular procedures. The substance comprises a volume of radiopaque marking substance suitable for creating a rapidly stable and precise image on a radiographic medical device when said material is placed externally on a patient and imaged by the radiographic medical device. The substance comprises a volume of forming material for combination with the radiopaque material. An associated dispenser is also disclosed.

### PRIORITY

The present application is related to, and claims the priority benefit of, U.S. Provisional Patent Application Ser. No. 63/100,222, filed Mar. 3, 2020, the contents of which are hereby incorporated into the present application directly and by reference in their entirety.

### FIELD OF THE INVENTION

The present disclosure includes disclosure comprising a substance suitable for external marking of a patient's skin and/or surgical covers or drapes as a procedural designated step (step-by-step surgical descriptions) during a medical imaging procedure, primarily a radiographic procedure, such as angiography, endovascular procedures, interventional radiology diagnostic and therapeutic procedures and any other procedure under fluoroscopy. The substance comprises a volume of radiopaque marking substance suitable for creating a rapidly stable and precise image on a radiographic medical device when said material is placed externally on a patient and imaged by the radiographic medical device. The substance comprises a volume of forming material for combination with the radiopaque material. An associated dispenser is also disclosed.

### BACKGROUND

### Prior Art

For decades, physicians have relied on external markings on patients to assist in site locations for surgical intervention using x-ray or radioscopy/fluoroscopy procedures. These systems built upon earlier marking technologies using radio-opaque solid metal markers or pointers made from, or perhaps painted with, a radio-opaque material, or by use of adhesive-backed tape carrying a radio-opaque substance, which has been placed on the part or parts of interest for industrial uses. Crayon-like markers as described in U.S. Pat. No. 4,813,062 capable of making marks on various substrates then evolved. Use of tape-like markers were described for use on cutaneous landmarks such as small bones of a foot in U.S. Pat. No. 5,193,106. In U.S. Pat. No. 5,469,847 the invention was an adhesive surface marker comprising a gel sealed by a casing and membrane structure. The markers provided means for marking patients and diagnostic images taken of those patients through different methods including x-ay, computerized tomography (CT), positron emission tomography (PET), and nuclear magnetic resonance imaging (NMRI).

Yet another skin marker for providing a reference point for a plurality of different medical imaging procedures is disclosed in U.S. Pat. No. 8,620,405. The marker incorporated one or more substances having one or more of radiance, hydration, radiopaque, radio luminescent and/or radioactive properties for detection by x-ray, CT, magnetic resonance imaging (MRI), ultrasonic scanning processes, or PET, and one or more markings recognizable by an optical imaging process such as 3D surface scanning.

U.S. Pat. No. 9,861,449 discloses marking implements for making radiopaque markings, and methods of making and using the same. The marking implements comprise a marking element formed of a composition comprising a carrier and a plurality of bismuth trioxide particles dispersed in the carrier. The composition is a thixotropic solid that maintains its shape at ambient temperatures but is deposited on skin at thicknesses between about 0.1 mm and about 1.5 mm when a shear force of between about 10N to about 35N is applied between the composition and the skin. The composition has an attenuation coefficient of greater than about 1,000 Hounsfield units (HU) at a thickness of about 0.6 mm for an x-ray having an incident intensity of about 135 kVp and about 1.5 mA. In some embodiments, the marking implements comprise a marking element formed of a composition comprising a carrier and a plurality of bismuth trioxide particles dispersed in the carrier, the carrier comprising about 35 wt % to about 50 wt % propylene glycol, about 10 wt % to about 15 wt % dipropylene glycol, and about 2 wt % to about 15 wt % sodium stearate. The disclosed marking implements could be used for a variety of applications.

In a non-medical related European Patent No. 0598468A1, a clear, water based polyvinyl alcohol adhesive gel is disclosed which contains a water-soluble high viscosity thickening agent polymer which provides thixotropic properties to the gel, a water-soluble plasticizer for the adhesive polymer, and a water-soluble defoamer. Due to its thixotropic properties, the viscosity of the adhesive will break down when a flexible tube or squeeze bottle is finger-pressed and has a sufficiently low viscosity to allow for extrusion from a small orifice such as one having a diameter of about 0.06 to 0.15 inches. When pressure is released after the desired amount of adhesive flows out of the orifice, the adhesive quickly reverts to very close to its original gel state so that a horizontal line of the gel will not run when applied to a vertical surface of a porous substrate such as paper.

Radiologists and endovascular surgeons have relied on injectable contrast material (ICM) to identify sites of interest or anomaly in patient vasculatures and organ systems. Such identification has been instrumental in guiding the physicians in subsequent invasive procedures and additional imaging. Typically, these procedures differ from x-ray, radioscopy and other imaging techniques in the high volume of ICM that is needed. Recently, there have been concerns expressed about potential adverse effects of such high volumes of contrast media, and components thereof, used in patients. See, for example, Investigation of the toxicity of bismuth oxide nanoparticles in various cell lines, by Abudayyak et al, http://dx.doi.org/10.1016/j.chemosphere.2016.11.018.

In general, there are two basic types of intravenous contrast material used for most radiological studies. They include ionic high-osmolality contrast media (HOCM) and non-ionic low-osmolality contrast media (LOCM). LOCM has become the preferred form of IV dye material in recent years, given its better safety record. However, it is more expensive than HOCM.

As such, the use of ICM to define vascular anatomy is necessary during medical imaging procedures. Unfortunately, ICM can cause serious cardiovascular reactions especially in patients with renal comorbidities. Hence, there is significant need to reduce the amount of ICM in medical imaging procedures to reduce contrast-related complications and cost.

### BRIEF SUMMARY

The present disclosure includes disclosure of a substance suitable for external marking of a patient's skin or the surgical covers or drapes, since it is more common to have all the patient and skin covered with single use disposable surgical sterilized covers during a radiographic procedure, such as angiography, endovascular and all other interventional radiology diagnostic and therapeutic procedures. The substance comprises a volume of radiopaque marking substance suitable for creating a rapidly stable and precise image on a radiographic medical device when said material is placed externally on a patient and imaged by the radiographic medical device. The substance comprises a volume of forming material for combination with the radiopaque material. An associated dispenser is also disclosed.

Exemplary devices of the present disclosure are configured to lower the overall costs related to major procedures, by decreasing the total volume of contrast needed and reducing procedure times. Reduction in ICM usage may also reduce the incidence of systemic complications related to contrast use therefore reducing hospital length of stay.

In at least one embodiment of an external medical marking substance for use on a patient during a medical imaging procedure of the present disclosure, the substance comprises a volume of non-toxic radiopaque material suitable for creating an image on a medical imaging device when said radiopaque material is combined with a forming material and the combination is placed externally on a patient and imaged by medical a imaging device, and a volume of forming material for combination with the radiopaque material, the forming material providing suitable characteristics for the combined materials forming the marking substance to create both an image visible to the naked eye and a radiographic image when the marking substance is viewed by the medical imaging device.

In at least one embodiment of an external medical marking substance of the present disclosure, the radiopaque material is selected from the list of radiopaque materials consisting of a mono-acidic monomer, a mono-acidic dimer, a non-ionic monomer, and a non-ionic dimer.

In at least one embodiment of an external medical marking substance of the present disclosure, the radiopaque material is a volume of inorganic metallic lead particles. In at least one embodiment of an external medical marking substance of the present disclosure, the volume of inorganic metallic lead particles comprises particles of more than one shape.

In at least one embodiment of an external medical marking substance of the present disclosure, the forming material is selected from the list of forming materials consisting of polyglucoside, hydroxyethylcellulose, carboxymethylcellulose, a skin adhesive formed from a polyvinylacetate base, and combinations thereof. In at least one embodiment of an external medical marking substance of the present disclosure, the forming material is selected from the list of forming materials consisting of carboxymethyl cellulose and polyglucoside ester.

In at least one embodiment of an external medical marking substance of the present disclosure, the marking substance is flowable for application onto a patient in a medical imaging procedure at temperatures between 15° C. and 50° C.

In at least one embodiment of an external medical marking substance of the present disclosure, the medical marking substance exits a dispenser as a gel suspension that rapidly dehydrates and hardens on the surface upon which it is deposited. In at least one embodiment of an external medical marking substance of the present disclosure, the hardening time after dispensing is between about 4 seconds and 10 seconds. In at least one embodiment of an external medical marking substance of the present disclosure, the viscosity of the marking substance is in a range of about 150,000 cP to about 250,000 cP. In at least one embodiment of an external medical marking substance of the present disclosure, the viscosity of the marking substance is in a range of about 150,000 cP to about 250,000 cP within 4 seconds to 10 seconds after exiting the dispenser.

In at least one embodiment of an external medical marking substance of the present disclosure, the concentration of the forming material in the medical marking substance is greater than about 40%. In at least one embodiment of an external medical marking substance of the present disclosure, the concentration of the forming material in the medical marking substance is between about 40% and 90%. In at least one embodiment of an external medical marking substance of the present disclosure, the concentration of the forming material in the medical marking substance is between about 75% and 95%. In at least one embodiment of an external medical marking substance of the present disclosure, the concentration of the forming material in the medical marking substance is between about 75% and 85%.

In at least one embodiment of an external medical marking substance of the present disclosure, the medical marking substance is suitable to create a line-like marking on the patient being imaged that has a thickness of between about 0.5 to about 1.5 mm. In at least one embodiment of an external medical marking substance of the present disclosure, the medical marking substance is suitable to create a line-like marking on the patient being imaged that has a width of between about 0.5 to about 1.0 mm. In at least one embodiment of an external medical marking substance of the present disclosure, the marking substance is non-toxic externally to the patient and is readily removed with water or a substantially neutral solvent.

In at least one embodiment of an external medical marking substance of the present disclosure, the substance is configured for use in connection with a dispenser, the dispenser comprising a central reservoir section configured to receive the medical marking substance, a first distal tip located at a first distal end of the dispenser, a second distal tip located at a second distal end of the dispenser, a first channel defined within the first distal tip, the first channel arranged in a first orientation, and a second channel defined within the second distal tip, the second channel arranged in a second orientation different from the first orientation. In at least one embodiment of an external medical marking substance of the present disclosure, the dispenser is configured for use in connection with a medical imaging device to place the medical marking substance externally on the patient and to visibly detect the medical marking substance as it is being dispensed by the dispenser.

In at least one embodiment of dispenser of a medical marking substance of the present disclosure, the dispenser comprises a central reservoir section configured to receive a quantity of a medical marking substance therein, a first distal tip located at a first distal end of the dispenser, a second distal tip located at a second distal end of the dispenser, a first channel defined within the first distal tip, the first channel arranged in a first orientation, and a second channel defined within the second distal tip, the second channel arranged in a second orientation different from the first orientation, wherein the dispenser is configured to dispense the medical marking material externally on a patient while the patient is undergoing a procedure using a medical imaging device. In at least one embodiment of dispenser of a medical marking substance of the present disclosure, the dispenser further comprises an extension element coupled thereto or formed as part thereof, the extension element configured to facilitate the dispensing of the medical marking material from a distance away from the dispenser so that a user's hand is not detected by the medical imaging device when dispensing the medical marking material.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 is a side elevation view of a contrast material dispenser of the present disclosure with distal caps covering both tips, according to at least one embodiment of the present disclosure;

FIG. 2 is an assembly view of one embodiment of a contrast media dispenser with distal caps removed from both tips and the tips removed from the reservoir section, according to at least one embodiment of the present disclosure;

FIG. 3 is a side sectional view of the contrast media dispenser with distal caps covering both tips, taken along line 3-3 of FIG. 1, according to at least one embodiment of the present disclosure;

FIG. 4 is an internal section view of the contrast media dispenser with distal caps covering both tips and showing the view from a mid-point of the reservoir toward one distal tip, as shown along line 4-4 of FIG. 3, according to at least one embodiment of the present disclosure;

FIG. 5 is a close up perspective view of a first distal tip, according to at least one embodiment of the present disclosure;

FIG. 6 is a close up perspective view of a second distal tip, according to at least one embodiment of the present disclosure;

FIG. 7A is a schematic of definition of region of interest (ROI), according to at least one embodiment of the present disclosure;

FIG. 7B is a demonstration of external marking pen as visualized under fluoroscopy, according to at least one embodiment of the present disclosure;

FIGS. 8A and 8B depict use of the marking material relative to the bodily features of interest, according to exemplary embodiments of the present disclosure;

FIG. 9 shows relative complication rates for using devices of the present disclosure as compared to conventional procedures expressed as percentage of total, according to at least one embodiment of the present disclosure; and

FIG. 10 shows a block component diagram of a dispenser of the present disclosure used in connection with an extension element, according to at least one embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

Many vascular and non-vascular pathologies in interventional radiology, such as cancer, uterine fibroids, renal/ureter obstructions, liver fibrosis and others may now be diagnosed and treated with minimally invasive procedures, attributable to advancements in interventional radiology. During the interventional procedure, the doctor may use Digital Subtracted Angiography (DSA), which are images showing the vessels, arteries and veins of a region or an organ, enhanced by injecting a radio-opaque contrast agent. In addition, catheter navigation and deployment of various tools are performed under visual feedback of real-time x-ray images, called fluoroscopy images (FI), but also with CT-scan and MRI imaging. These images show contrast located in the imaged region of interest, such as vasculature in an organ or in a malign or benign tumor internally located in a patient's body. However, the patient's vessels are only briefly visible in the sequence because contrast material rapidly flows through and out of these vessels.

To show the tools in relationship to the patient's vasculature for a large period of time, real time fluoroscopy images are subtracted from a mask image of the vessels acquired using more contrast medium for this purpose. This process is referred to as a Roadmap. Despite its benefits, the Roadmap process also presents well-known limitations, such as: (1) requiring an extra injection of large amounts of contrast media each time a roadmap is needed, thereby increasing the total contrast media injection into the patient's circulation, (2) the Roadmap does not provide three dimensional information, so the navigation within the vasculature requires a mental reconstruction of the patient's vessels and tools; (3) any change in the imaging device, e.g. C-arm orientation and table position, makes the Roadmap flawed and requires a further injection of contrast agent; and, (4) the image quality is reduced compared to DSA.

Considering that patients undergoing any type of an interventional radiology procedure have at least one moderate to severe disease, but most often present with other systemic disorders, it is important to avoid any complication related to the methods used in this field. Unfortunately, one of the most common complications related to contrast media injections is acute renal failure and thrombosis. Other complications can be characterized by severity and type of symptoms.

Mild symptoms include scattered urticaria (which is the most commonly reported adverse reaction), pruritus, rhinorrhea, nausea, brief retching and/or vomiting, diaphoresis, coughing and dizziness. Patients with mild symptoms should be observed for the progression or evolution of a more severe reaction, which may require treatment.

Moderate symptoms include persistent vomiting, diffuse urticaria, headache, facial edema, laryngeal edema, mild bronchospasm or dyspnea, palpitations, tachycardia, or bradycardia, hypertension and abdominal cramps.

Severe symptoms include life-threatening arrhythmias (e.g., ventricular tachycardia), hypotension, overt bronchospasm, laryngeal edema, pulmonary edema, seizures, syncope, and death.

Nonidiosyncratic reactions include bradycardia, hypotension, vasovagal reactions, neuropathy, cardiovascular reactions, extravasation, and delayed reactions. Other nonidiosyncratic reactions include sensations of warmth, a metallic taste in the mouth, and nausea and vomiting. With regard to the bradycardia, hypotension, and vasovagal reactions, there is a heightened systemic parasympathetic activity that is induced. The injected contrast media (ICM) can actually precipitate bradycardia (e.g., decreased discharge rate of the sinoatrial node, delayed atrioventricular nodal conduction) and peripheral vasodilatation. The end result is systemic hypotension with bradycardia. This may be accompanied by other autonomic manifestations, including nausea, vomiting, diaphoresis, sphincter dysfunction, and mental status changes. Untreated, these effects can lead to cardiovascular collapse and death. Some vasovagal reactions may be a culminating result of coexisting circumstances such as emotion, apprehension, pain, and abdominal compression, rather than solely the ICM administration.

Contrast agent-related nephropathy is an elevation of the serum creatinine level that is more than 0.5 mg % or more than 50% of the baseline level at 1-3 days after the ICM injection. The elevation peaks by 3-7 days, and the creatinine level usually returns to baseline in 10-14 days. The incidence of contrast agent-related nephropathy in the general population is estimated to be 2-7%. As many as 25% of patients with this nephropathy have a sustained reduction in renal function, most commonly when the nephropathy is due to reduced secretion of urine, i.e. oliguric. The mechanism of this type of nephropathy is thought to be a combination of preexisting hemodynamic alterations, renal vasoconstriction—possibly through mediators such endothelin and adenosine, and direct ICM cellular toxicity.

As noted above, injected contrast media may cause serious cardiovascular reactions such as hypotension and bradycardia. Vasovagal reactions, a direct negative inotropic effect on the myocardium, and peripheral vasodilatation probably contribute to these effects. The latter two effects may represent the actions of cardioactive and vasoactive substances that are released after the anaphylactic reaction to the ICM. This effect is generally self-limiting, but it can also be an indicator of a more severe, evolving reaction. ICM may also lower the ventricular arrhythmia threshold and precipitate cardiac arrhythmias and cardiac arrest. Fluid shifts due to an infusion of hyperosmolar intravascular fluid can produce an intravascular hypervolemic state (i.e., excess fluid in the blood), systemic hypertension, and pulmonary edema. Also, ICM can precipitate angina.

Moreover, the similarity of the cardiovascular and anaphylactic reactions to injected contrast media can create confusion in identifying the type and severity of an adverse reaction. This confusion can lead to the overtreatment or undertreatment of symptoms. Other nonidiosyncratic reactions include syncope; seizures; and the aggravation of underlying diseases, including pheochromocytomas, sickle cell anemia, hyperthyroidism, and myasthenia gravis.

Another concern relating to ICM is extravasation into tissue. Extravasation of ICM into soft tissues during an injection can lead to tissue damage as a result of direct toxicity of the contrast agent or pressure effects, such as compartment syndrome. Delayed reactions also occur, and become apparent at least 30 minutes after, but within 7 days of, the ICM injection. These reactions are identified in as many as 14-30% of patients after the injection of ionic monomers and in 8-10% of patients after the injection of nonionic monomers. Common delayed reactions include the development of flu-like symptoms, such as fatigue, weakness, upper respiratory tract congestion, fevers, chills, nausea, vomiting, diarrhea, abdominal pain, pain in the injected extremity, rash, dizziness, and headaches. Less frequently reported manifestations are pruritus, parotitis, polyarthropathy, constipation, and depression.

To avoid any of these adverse reactions, complications and very costly hospital admittances related to injected contrast media, it is important to lower the amount of contrast media injected into the patient's circulatory system. One of the options available is to decrease the need for Roadmap imaging and/or contrast injections to “check” catheters, balloons and stent positioning. The invention enables lowering the volume of contrast media injected into patients. This has significant impact in the endovascular field for procedures such as diagnostic and therapeutic angiography, angioplasty and stenting, treatment of venous and arterial thromboembolic disease, aortic aneurysm repair, inferior vena cava filter placement, vertebroplasty, thoracocentesis and paracentesis, peripheral vascular bypass, carotid angioplasty and stenting, dialysis access, thrombolytic therapy, and others.

One example of the potential for reduction of injected contrast material was assessed by reviewing one year of diagnostic and endovascular therapeutic procedures in a large Sao Paulo, Brazil, hospital. The total number of procedures was 323. Of this group, 108 patients underwent diagnostic exams (33.4%) using a total of 15,214 ml of injected contrast material (M=140.5 ml). 215 patients had therapeutic procedures (66.6%), using a total of 37, 961 ml of injected contrast material (M=176.5 ml). The total volume of injected contrast material used that year was 53,175 ml (M=164.6 ml), at a total cost of $116,297 USD (i.e., diagnostic $38,886 and therapeutic $77,412). It should also be understood that the initial injection of contrast media that is used for initial visualization is about 15 ml to 30 ml. However, additional injections typically follow. With the use of the current invention, the initial volume may go down significantly and it may be possible to eliminate subsequent injections, subject to procedural and patient parameters.

Non-vascular disorders are also treated with interventional radiology procedures, using minimally invasive techniques that may also have applicability to the use of the invention in procedures such as neoplasic tumor feeding arteries and vein locations, embolization, immunotherapy, chemoembolization, ablations and other procedures.

The invention is also developed to lower the overall costs related to major procedures, in the cardiac, vascular and neurologic fields, because it will decrease the total use of volumes of expensive contrast material. Similar savings may occur through reduced procedure time leading to lower operating room or angiosuite expenses. Patient stays in hospitals, particularly those that develop multi-systemic complications related to the use of high volumes of contrast, may also be reduced. In the same patient review discussed above, 28% of patients undergoing a vascular procedure developed elevated creatinine levels indicative of renal failure, i.e. greater than 1.75 (normal baseline=1.00). A similar elevation of creatinine levels occurred with 19% of cardiology patients.

In view of financial savings and the desire to reduce exposure of patients to unnecessary contrast media, there is a goal of the present invention to substantially reduce the number and volume of contrast injections, particularly the high-volume intravascular injections associated with angiography and endovascular procedures. Accordingly, the invention teaches a method for reducing the internal exposure of patients to radiographic contrast material in a medical imaging device procedure. The method steps include: a) providing an external medical marking substance for use on a patient, and the external medical marking substance comprising a volume of radiopaque material and a volume of forming material; b) configuring the volume of external medical marking substance to have rapid setting properties and being suitable for creating a radiographic image of a patient during a radiographic procedure when the marking material is placed externally on the patient while being imaged by the radiographic medical device; c) configuring the volume of external medical marking substance to have rapid setting properties suitable for creating both an image visible to the naked eye and a radiographic image as the marking substance is viewed by the medical imaging device, and d) applying the external medical marking substance on a patient to identify dynamic organ functions of the patient derived from an initial internal contrast injection into the patient during an imaging procedure and then applying the marking substance during the visual pendency of the initial contrast injection. This goal is achieved through use of a gel-like material that creates a stationary layer (also containing radiopaque contrast media) over select portions of the skin and/or surgical covers of patients. The stationary layer material is non-toxic, non-allergenic, and readily removable at the completion of the medical procedures. The formula of the material should be radiopaque enough to allow the surgeon to visualize markings with the marking substance (and designated dispensing tips on a preferred dispenser) as a line or an area in a real-time manner during the procedures, after initial visualization has been achieved with the first volumes of intra-vascular contrast material.

The invention involves an external medical marking substance for use on a patient during a medical imaging procedure, and a device suitable for dispensing the substance during the procedure. Preferably, the use of the substance reduces the volume of potentially toxic or allergenic contrast media that a patient is exposed to internally. Use of the marking substance of the invention will materially reduce the total injected contrast material volume for numerous invasive ICM procedures, depending on the procedure being performed.

In one embodiment, the external medical marking substance comprises a volume of radiopaque material suitable for creating an image on a radiographic medical imaging device when the material is placed externally on a patient and imaged by the medical imaging device. The radiopaque material is precisely positioned on the patient using a volume of forming material combined with the radiopaque material. The forming material provides suitable characteristics for the combined materials so that the marking substance creates both an image visible to the naked eye and a radiographic image by the medical imaging device. The radiopaque material is selected from the list of radiopaque materials consisting of ionic high-osmolality contrast media, non-ionic low-osmolality contrast media and iso-osmolality contrast media. These may be water-based or oil-based, and may be selected from a list of radiopaque materials consisting of a mono-acidic monomer, a mono-acidic dimer, a non-ionic monomer, and a non-ionic dimer.

The radiopaque material may be selected from the list of radiopaque materials consisting of, based upon or referred to as: diatrizoate (sold under tradenames Hypaque, Urograffin, and Angiograffin), diatiazic acid, metrizoate (sold under tradenames Isopaque and Coronar), metrizamide, iodamic acid, iothalmic acid, ioxithalmic acid, iothalamate (sold under tradename Conray), iohexol (sold under tradename Omnipaque), iodamide, iopromide (sold under tradename Ultravist), ioglicate, iocarmic acid, ioglicic acid, iodixanol (sold under tradename Visipaque), iotrol, ioxithalmate, ioversol (sold under tradename Optiray), iopentol, ioxilan, iorrol, iopamidol (sold under tradenames Isovue, Lek-Pamidol and lopamiron), iotrolan, gadolinium (sold under tradename Gadavist), iodipamide, gadodiamide (sold under tradename Omniscan), gadolinium-based, manganese-based, iron-based, gadobenate dimeglumine (sold under tradename Gadoxetate), iodine-based, non-iodine-based, ioxaglate (sold under tradename Hexabrix), ioxaglate, ethiodol, pantopaque, lipidol, salts of sodium and meglumine, meglumine (sold under tradename Dotarem), salts of sodium and meglumine with tri-iodinated fully substituted benzoic acid ring (sold under tradename Sinograffin), dimeglumine (sold under tradename Primovist), super paramagnetic iron oxide (SPIO) particles (sold under tradenames Endorem and Feraheme), inorganic metallic lead, bismuth, titanium, tungsten, patient blue violet, simethicone-coated cellulose, natural bone mineral material, processed animal bone material, processed bovine bone material, a phosphate mineral from the apatite group, hydroxyapatite, synthesized hydroxyapatite, ultrasonically synthesized hydroxyapatite, nano-crystalline hydroxyapatite, microcrystalline hydroxyapatite, biphasic calcium phosphate, tricalcium phosphate, biphasic tricalcium phosphate, and β-tricalcium phosphate. It is understood that the above is a representative list and exemplary tradenames (and names of compound formulations used to achieve various X-ray attenuation levels and other characteristics) without limiting the use of analogs or similars under any other tradenames or compound formulation names with similar formulations.

The medical marking substance may be in the form of a suspension, a semi-solid, a liquid, a gel, an aerosol, or a phase-changing formulation. The medical marking substance may have Newtonian or non-Newtonian flow characteristics. In one embodiment, the marking substance is flowable at temperatures between 17° C. and 50° C., or substantially in the combined range of the operating temperatures of the medical procedure suites as well as the associated equipment. The medical marking substance may commence a phase shift to a gel when exposed to ambient atmosphere or when in a temperature range of about 15° C. to about 32° C. in about 8 seconds. The medical marking substance may have thixotropic properties to enable storage and dispensing, as well as stability at dispensed sites on patient skin or other tissue, or onto a material covering part of the patient.

In one embodiment, the forming material is made from one or more of a thickening agent, a buffer, stabilizers, and one or more preservatives. In one embodiment, a buffer phosphate USP having a pH of about 6.8 was used with stabilizers such as carboxymethyl cellulose (commonly referred to as CMC) or polyoxyethylene sorbitan monooleate (commonly referred to as polysorbate 80 or tradename Tween 80). Parabens was used as a microbial preservative. Additional materials may include black iron oxide, titanium dioxide and metallic inorganic lead.

In another embodiment, the forming material is selected from the list of forming materials consisting of polyglucoside, hydroxyethylcellulose, and an adhesive-like material formed from a polyvinylacetate base, and combinations thereof. Regardless of the forming material selected, it is preferred that the forming material demonstrate's rapid setting properties and proper viscosity parameters to ensure precise lines and shapes when seen under imaging. Regardless, the marking substance must also be non-toxic when applied externally to a patient and must be readily removed with water or a substantially neutral solvent when directed. Preferrably, the marking substance should be compatible with both mammalian skin and surgical drape materials. The medical marking substance should preferably have a viscosity in a range of 150,000 to 250,000 cps (centipoise) after application onto a patient within 4 to 10 seconds. These attributes are not easily achieved, particularly when the marking substance is applied during a medical procedure.

In one embodiment using a mild adhesive formed from a polyvinylacetate base, the chemistries are favorable but the rapid setting/curing of the forming material and the formation of the radiopaque material is not optimal. In a more preferred embodiment, use of a polyglucoside ester improves the overall outcome in an unexpected manner. Upon further research it was determined that the polyglucoside ester has a more accelerated water evaporation factor, thereby producing a more rapid and thicker marking substance. This also enables more favorable management of the radiopaque material in the marking substance—contributing to the precision and enhanced visibility of markings on the patient when viewed under medical imaging devices. This is also a favorable characteristic from the perspective of a clean surgical field. The rapid setting of the marking substance, which itself is a sterile material from a single use dispenser, also ensures that the substance will minimize capture of any unwanted foreign material in the substance on a patient.

The marking substance comprises a volume of forming material for combination with the radiopaque material. Preferably, the concentration of the forming material in the marking substance formulation is greater than about 40%. Alternatively, the concentration of the forming material may be between about 40% and 90%. In another embodiment, the concentration may be between about 75% and 95%. In a further embodiment, the concentration may be between about 75% and 85%.

The forming material may also comprise a thickening agent, or agents. The radiopaque material may comprise different concentrations and sizes of metallic inorganic lead to optimize imaging, as desired.

The marking substance is formed of radiopaque materials that allow for both visual and radiographic marking of patients for use during interventional radiology diagnostic and therapeutic procedures where visible marking is beneficial. The marking substance is a non-toxic composition that can easily be applied in a thin, even and consistent layer, formed of a composition of a forming substance (carrier), a plurality of radiopaque particles with different diameters and concentrations, and a colorant. The marking composition maintains its shape stable and semi-solid (gel) when deposited on skin or surgical covers/drapes at ambient temperatures between 15° C. and 32° C. with a thickness between 0.5 to 1.5 mm, a hardening time between 4 to 10 seconds, and produces a drawing line width between 0.5 to 1.0 mm, or a brush area when needed. At any thickness and width, the marking substance will have an attenuation coefficient greater than about 1,000 HU (Hounsfield units) for an X-ray intensity of about 135 kVp and about 1.5 mAs.

In one embodiment, the viscosity of the marking substance using a polyglucoside forming material was determined using a Brookfield Dial Reading Viscometer device with an LV4 spindle at 2 rpm. The torque was approximately 45% to 55% at 25° C., generating a range of 150,000 to 250,000 centipoise (cP).

A preferred embodiment of the marking substance is not absorbed by skin, does not act as a depot delivery mass after deposition on the patient's skin, and does not produce any residual material after proper removal from the surface area onto which it was deposited. The marking substance is substantially inert with skin, glass or plastic, and does not penetrate the skin beyond the stratum corneum upper layer of the epidermis.

The embodiment of marking substance comprising a radiopaque material of inorganic metallic lead particles is also non-penetrating. Even if the inorganic metallic lead were to be directly placed on skin, which it is not, it has a very low absorption through skin (less than 0.06% in chronic exposures) and a blood level of less than 0.05 ug/dl. Rather, the inorganic metallic lead particles are in the forming material suspension, and are retained in the suspension. This retention is both prior to and after the approximately 8 second setting time for the marking material after placement on the patient. One advantage of this embodiment of marking substance is that even though the most radiopaque organ in the patient's body are the bones, the marking substance is visible even on top of these sites. The forming materials are all used in a daily basis with skin products and have been already classified as non-toxic products.

When applied on the skin with a minimum thickness of 0.5 mm, the preferred marking substance creates a darker static image than the internal ICM. Moreover, injectable contrast material has two characteristics that make it less useful than the skin-deposited radiopaque marking substance: 1) it may not have sufficient ICM injection thereby reducing its visibility under fluoroscopy and making additional injections needed, and 2) the ICM is removed from the area with blood flow making it visible only for a short period of time, and not leaving any residual marking substrate suitable for procedural guidance.

In certain patients with known allergies such as atopy (eczema) and/or specific dermatologic allergies and patients with underlying open skin lesions like ulcers, traumatic or surgical wounds, the marking substance should be used on the surgical covers/drapes, instead of directly on the skin. In patients with no allergic history but with open wounds, the marking substance can be used within a safe distance of the wound (at least 5 cm-10 cm) or equally on top of the surgical covers/drapes.

Sterilization of the marking substance and the device disclosed herein can be achieved by a one, or a combination of, heat, chemicals, irradiation, high pressure and filtration techniques. Effective sterilization techniques are essential for working with surgical and medical devices in the operating room and angiosuites, and are well known in the field. It is likely that the preferred sterilization technique to be used with the marking substance and its container will be gamma-ray methods of sterilization.

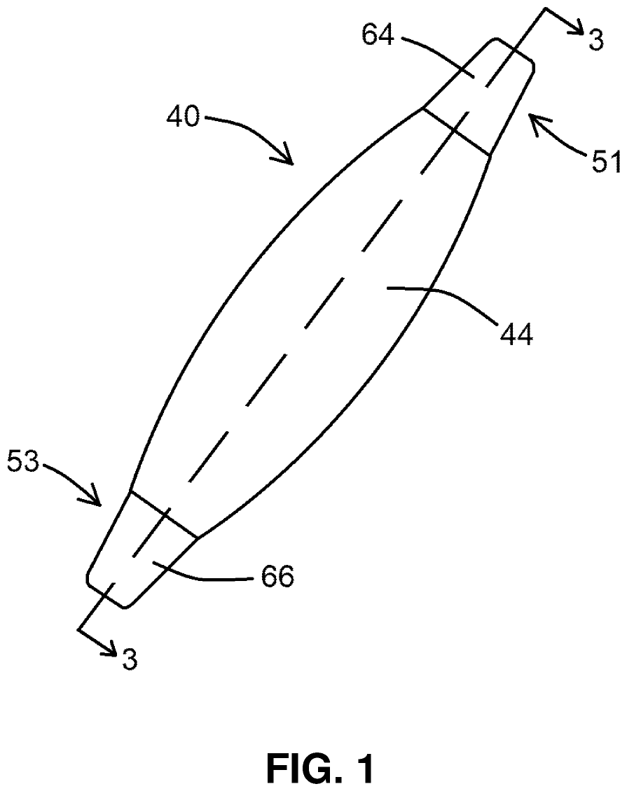

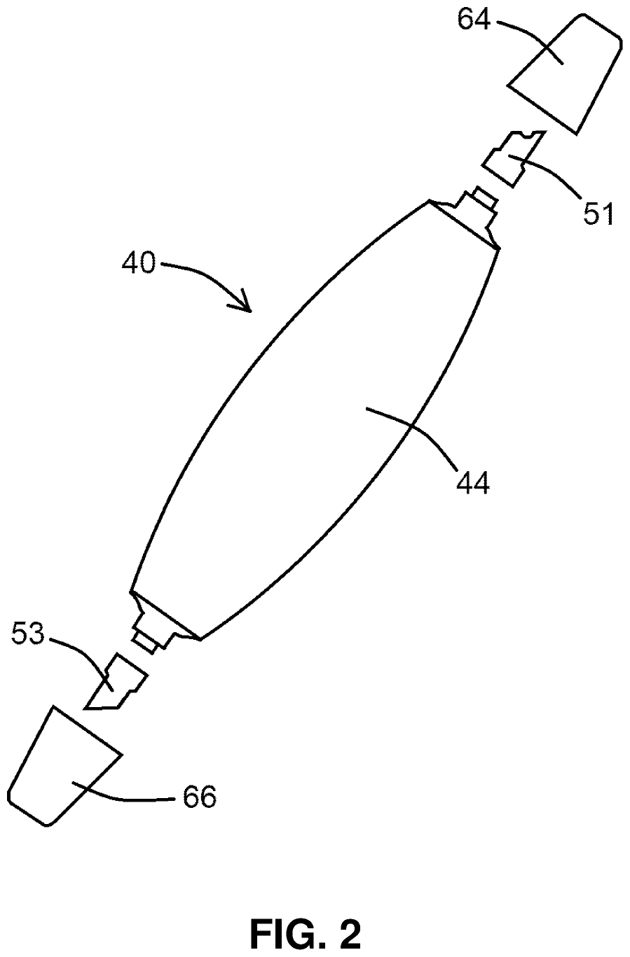

FIGS. 1 and 2 disclose one embodiment of a marking material dispenser 40, having a central reservoir section 44, dual dispensing tips 51, 53, and dispensing tip caps 64, 66. In this embodiment, there are tips provided that enable different amounts and shapes of marking material to be dispensed through the tips 51, 53. In this embodiment, it is preferable to have the dispenser components made of a plastic or plastic-like material that allows compression of the central reservoir section that in turn causes release of marking material from the reservoir and through a selected distal tip. It is recognized that the distal tip structures shape lines or patterns of released marking material according to the aperture size and shape of the aperture and tip. Numerous aperture and tip sizes, shapes and configurations are contemplated, and the modular structure enables ease of assembling different tip configurations as desired during imaged surgical procedures.

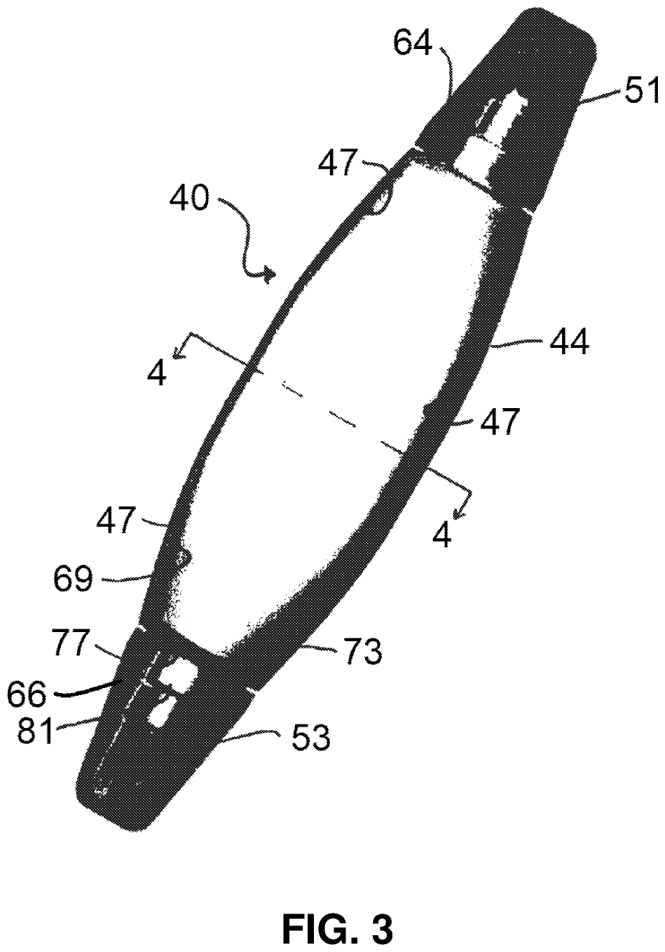

As shown in FIG. 3, there two distal dispensing tips that are configured for selective use by the medical professional using the dispenser 40. Reservoir section is designed to receive and store a volume of marking material, as described herein. Also shown are reservoir section walls 47 formed preferably of resilient plastic material to create the void that receives the marking material. Walls 47 enable finger and hand pressure to be applied to the reservoir section to form sheer stress on marking material within the reservoir section.

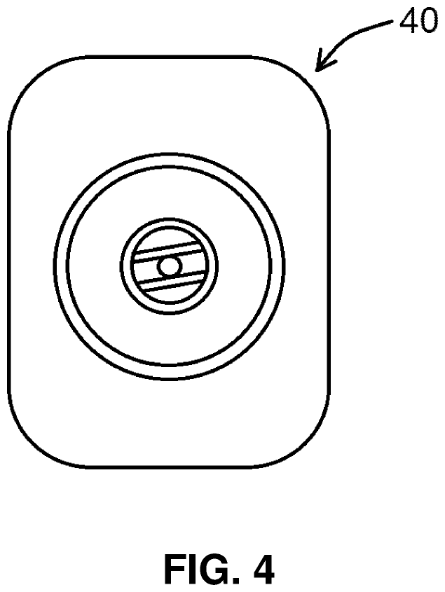

FIG. 4 is a sectional view of the internal structure of dispenser 40 taken along line 4-4 of FIG. 3. This view shows the internal wall section variable tapering sections 69, 73, 77, and 81 intended to impart various sheer forces on the marking material within the dispenser. As will be further disclosed below, these internal structural surfaces facilitate the conversion of the thixotropic contrast material into a semi-solid gel for delivery through the distal tips 51, 53 and out the distal tip apertures formed by aperture walls 87.

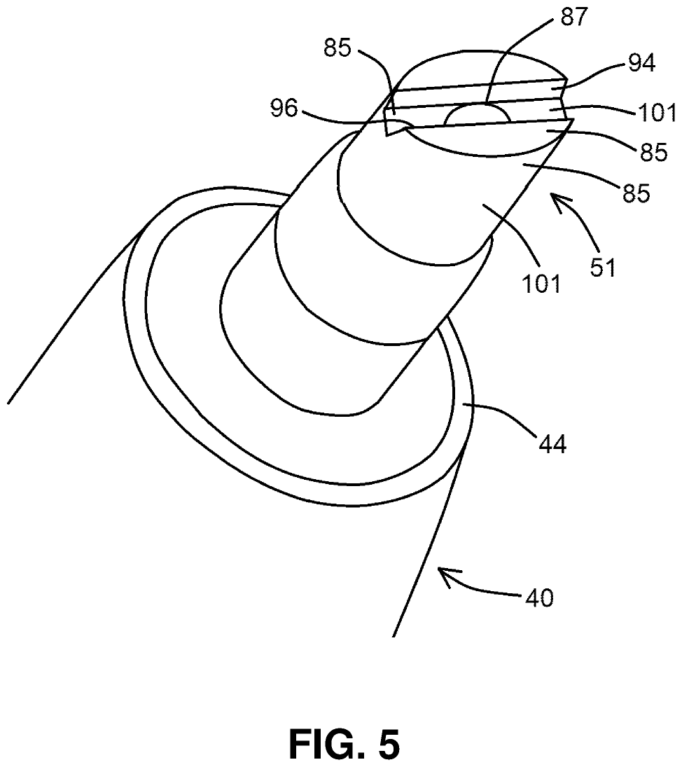

FIG. 5 shows a first distal tip 51 detail structured with applicator material 85 suitable for shaping dispensed marking material. Distal tip is made of plastic or metallic material with the end of the cannula shaped with side channel walls and the channel base where the cannula communicates to the dispenser. The cannula diameter varies from 0.5 to 1.5 mm, to allow very fine lines to be used in procedures dealing with small diameter vessels and the large diameter to allow its use to mark not only lines but also entire fields where the procedure will be done. A brush tip is also described for the same purpose.

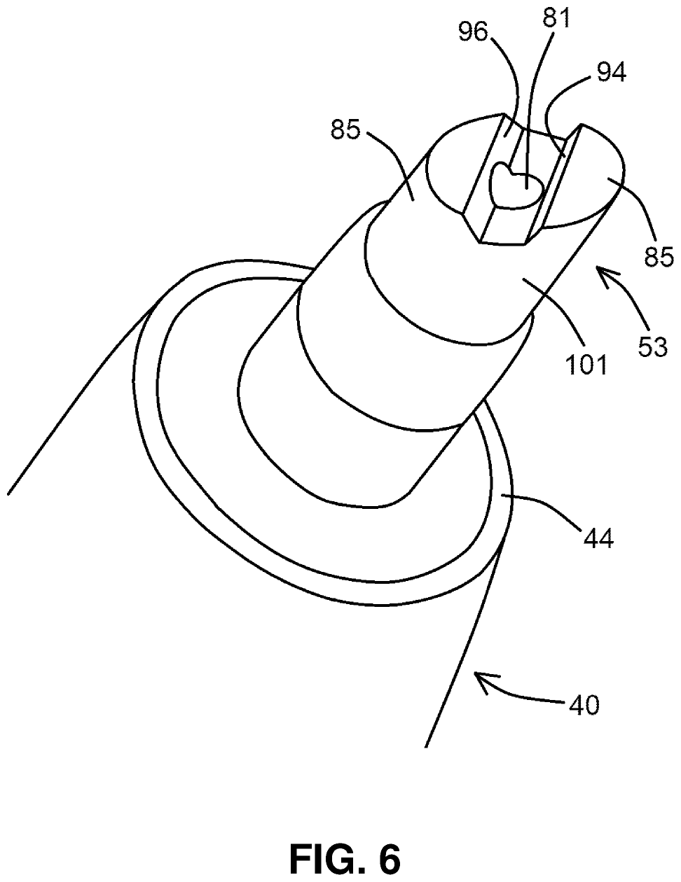

FIG. 6 shows the second distal tip 53 with applicator material 85 suitable for shaping dispensed marking material. As previously noted, dispensing tip structures may vary to achieve deposition of the marking material as needed. In FIGS. 5 and 6, the tip structures each comprise a first side channel wall 94 and a second side channel wall 96, and each tip also has a channel base structure 101. In this embodiment, the tip structures formed by walls and base structures create a channel that is oriented at a 90° angle between the two distal tips. This enables ease of use of the dispenser as needed by the user. Other tip structure configurations are possible and fully functional under this invention.

In at least one embodiment, the formation material used comprises a polyglucoside ester greater than 40% of the overall marking substance. Alternatively, the concentration of the forming material may be between about 40% and 90%. In another embodiment, the concentration may be between about 75% and 85%. In a further embodiment, the concentration may be between about 75% and 95%.

In various embodiments, the radiopaque material comprises inorganic metallic lead particles suspended in the formation material at a concentration less than 60% of the overall marking substance. In some embodiments, the medical marking substance may commence a phase shift to a gel when exposed to ambient atmosphere or when in a temperature range of at or about 15° C. to or about 32° C. in about 8 seconds or so (such as 8+/−2 seconds). In various embodiments, the medical marking substance should preferably have a viscosity in a range of 150,000 to 250,000 cps (centipoise) after application onto a patient within 4 to 10 seconds.

A benefit to using both forming materials of the present disclosure is that they do not penetrate bellow the epidermic cornea (Keratin layer). In at least some embodiments, the forming material is a suspension and the radiopaque material mainly formed of inorganic metallic lead particles with different diameters and concentrations.

According to the present disclosure, the relative duration of the marking material contact with the patient is very short, and as the stability of the exemplary marking suspension in the forming material of the present disclosure will prevent migration (such as, for example, like a depot drug delivery mechanism) into the skin. When the setting or curing into a semi-gel occurs then this further prevents lead migration

In at least some embodiments, the ink (marking material or marking substance) can be erasable in the context of interventional procedures as with disclosed composition or more permanent in case of oncology applications (e.g., breast tumor, etc.) to demarcate a tumor for surgeon to resect. In the latter, “glue” (e.g., cyanoacrylate) can be added to adhere to skin and make it less erasable at least for matter of days or few weeks.

To avoid the hand to be placed under x-ray panel during drawing on skin or covers, an extension element 110, such as that in a form of long syringe or piston, is used and configured to allow a user to push the ink through the pen tip. Such an extension element 110 is shown in block component format as being operably coupled to dispenser 40 in FIG. 10.

Further, and so that this technology is applicable to various vessel or structure sizes, the present disclosure includes disclosure of different distal tip 51, 53 sizes, whereby a relatively smaller distal tip 51, 53 could be used in connection with a smaller vessel or structure size, and whereby a relatively larger distal tip 51, 53 could be used in connection with a larger vessel or structure size. To account for changes in size, ink (marking material or marking substance) can have different viscosities, such as a relatively less viscous material used with a smaller distal tip 51, 53, and such as a relatively more viscous material used with a larger distal tip 51, 53. For example, lymphatic applications include smaller vessels while aortic applications include larger vessel. The viscosities can be tuned through changes in compositions as may be desired.

### Example Marking Material Formulation

An exemplary marking material of the present disclosure is shown in Table 1 below, noting that the exact amounts are not the only amounts able to be used in connection with various formulations applicable to the present disclosure:

TABLE 1
Example ingredients for formulation

| ELEMENT | % P/P | Kg × 100 Kg lot |
|---|---|---|
| Micronized Lead powder 100% | 13.0 | 13.200 |
| Tantalum | 1.2 | 1.200 |
| Polysorbate 80 | 1.9 | 1.900 |
| Propylene glycol | 4.7 | 4.700 |
| Methylparaben | 0.095 | 0.095 |
| Propylparaben | 0.047 | 0.047 |
| Carboxymethyl cellulose Na | 2.94 | 2.940 |
| Titanium dioxide | 0.95 | 0.950 |
| Black iron oxide | 2.84 | 2.840 |
| Phosphate buffer solution USP ph 6.8 | 72.328 | 72.328 |
| TOTAL | 100.0 | 100.000 |

### Methodology

The present process is for the production of a buffer solution for 72.558 Kg with the exceeding amount being calculated for water evaporation. The goal of this formulation was to create a solution with a texture of a semi firm gel.

Step 1: In a stainless steel tank smoothly stirred between 30 and 100 rpm, 493.2 g of Monopotassium phosphate p.a quality. Dissolve in 72.0 kg of distilled water. After the salt is completely dissolved into the water. Add easily and without stopping the stirring movement a total of 65.0 g of sodium hydroxide p.a quality. After the formation of a transparent solution check the pH, which should be between 6.6 and 7.0.

Reactor 1: In stainless steel with heating shirt to a minimum 80 C and cooling up to 25 C, with a 250-300 kg capability and dented grinding disc with a rotation ratio from 30 to 3000 rpm implemented.

Add to Reactor 1:

| | |
|---|---|
| Buffer Phosphate USP pH 6.8 | 72.328 Kg |
| Propylene glycol | 4.70 Kg |
| Polysorbate 80 | 1.90 Kg |
| Methylparaben | 0.095 Kg |
| Propylparaben | 0.047 Kg |

Apply heat evenly and thoroughly to 60 C and stir at approximately 30-50 rpm until total Polysorbate 80 is dissolved, resulting in a yellowish transparent solution.

Add:

| | |
|---|---|
| Titanium dioxide | 0.950 Kg |
| Black iron oxide powder | 2.840 Kg |

Stir to 100-150 rpm until black iron particles rise to the surface of the mixture. Then cool mixture to 30 C approximately. Shut off temperature regulation and keep mixture at (room temperature).

Step 2: At room temperature of a maximum 30 C, add:

| | |
|---|---|
| Micronized Lead powder 100% | 13.0 Kg |
| Tantalum | 1.2 Kg |

Stir to 150-300 rpm until the Lead comes up to the surface of the mixture.

Step 3: Sodium carboxymethylcellulose 2.940 Kg Separate the Sodium Carboxymethyl cellulose in to five individual portions of 588 g each for a total of 2.940 Kg. Add one 588 g individual portion into the solution one at a time until throughly mixed. All five of the 588 g portions will have to be mixed in to the solution for the total of 2.940 Kg of Sodium Carboxymethyl. Note: In adding each 588 g portion, an increase of the solution's viscosity should be observed. It is then necessary to gradually increase the rotation speed to 1000 rpm. Then up the speed to 1500 rpm to 2000 rpm and then up to 3000 rpm. More speed if necessary. During the mixing/stirring process, a dispersion-dissolution vortex around the mixing/stirring axel should be noticed and will assist you on knowing when to speed up the rotation.

Step 4: After the last, of the five 588 g portion is added, proceed with maximum rotation velocity while observing the vortex phenomenon previously reported. Remain at current pace until a fixed viscosity level is noticed, in which there are no carboxymethylcellulose lumps undissolved. To make sure, take a sample and spread on a glass surface observing if any lump appears when gently pressed with a spatula. The final result should be a clean uniform black paint like mixture.

Step 5 (Alternative): If for some reason the level of uniformity is not reached, then the mixture should pass through an homogenizer or a Zirconia beads pearl mill, which should fix the problem with 1-4 run's through it.

NOTE: Whether step 4 makes itself necessary or not, it will depend on the mixing/stir potency in Step 1. It is most important to make sure to have a pretty potent reactor, or else, auxiliary equipment will make itself necessary because of the high viscosity of the marking gel (which is around 150 k to 250 k cps at 25 C). Step 5, was not necessary at the development laboratory with quantities up to 400 g. In industrial scale quantities with over a 100 Kg, the solution gel should be submitted to test.

Step 6: Final product Quality Control approval

On a 300 g sample of the final product, the following tests must be run according to 148 MPA methodology attached

1. Aspect: High viscosity gel, low fluidity, black colored and lump free

2. Brookfield Viscosity with LV4 Needle at 2 rpm with approximately 45%-55% torque at 25 C.

Data-sheet: 150 k-200 k cps.

3. Direct gel pH with glass membrane at 25 C.

Data-sheet: 6.5-7.0

4. Density at 25 C through heavy beaker.

Data-sheet: 1.17-1.50 g/ml

5. Centrifugation: In a tube centrifuge with 7 ml of gel at 7000 rpm during 30 minutes at 25 C Data-sheet: maximum liquid separation must be less or equal to 1 nil.

Step 7: Fractioning After CQ Final Product approval, it proceeds to the primary gel packaging, hereafter with the corresponding secondary packaging.

### Patient Study

As discussed further herein, the present disclosure includes disclosure of the development of an external radiopaque marking device (referred to herein as dispenser 40) to lower the overall costs related to major procedures, by decreasing the total volume of contrast needed and reducing procedure times. Reduction in ICM usage may also reduce the incidence of systemic complications related to contrast use therefore reducing hospital length of stay. The endpoints of the present method study were to compare 1) the total the volume of contrast used, 2) overall costs, and 3) complications between the group of patients submitted to current/conventional therapeutic endovascular methods without using the external marking pen, and the group of patients undergoing endovascular therapeutic interventions using the marking device (dispenser 40).

### Methods

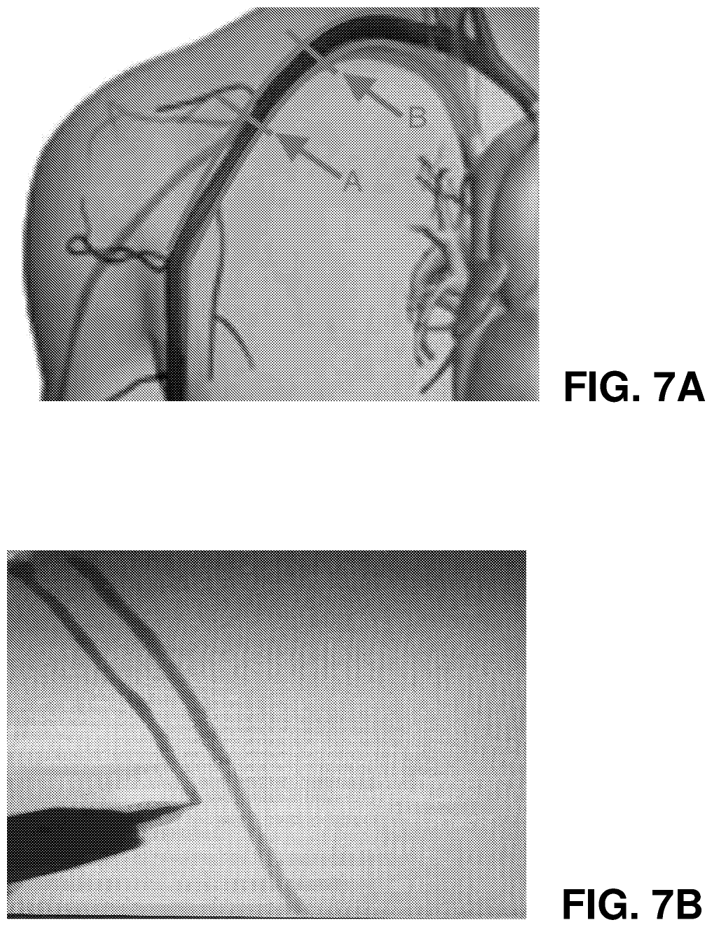

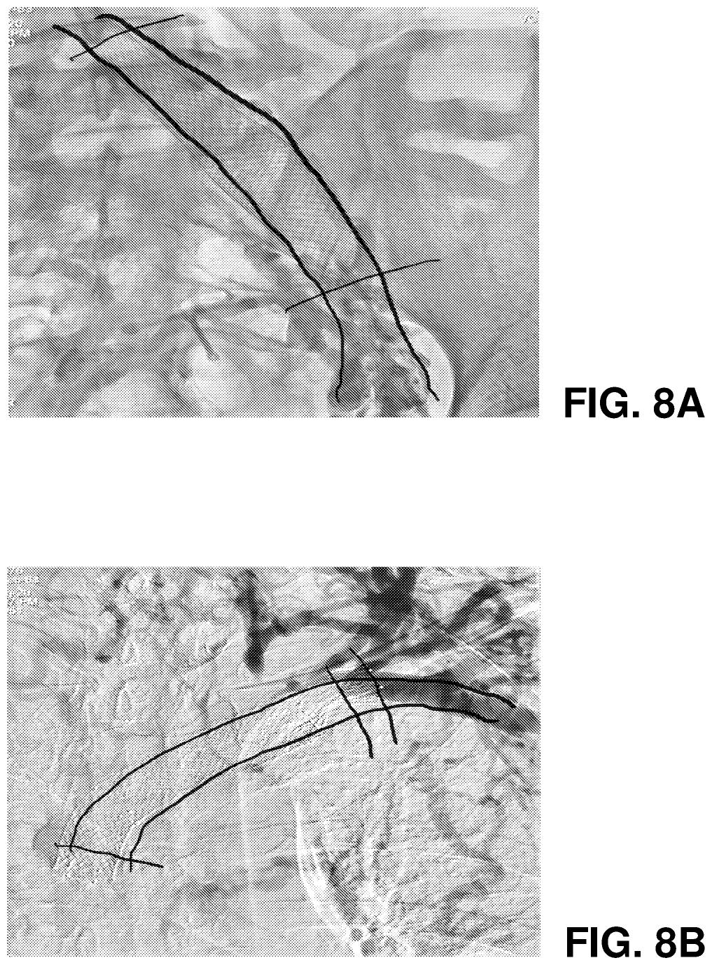

The steps for the dispenser 40 procedure include providing a sterilized external medical marking substance for use on a patient. The external medical marking substance consists of a volume of radiopaque material and a volume of forming material with rapid setting properties suitable to create a radiographic image during a radiographic procedure when the marking material is placed externally on the patient while being imaged by the radiographic medical device. The marking is visible to both the naked eye as well as a radiographic image as the marking substance is viewed by x-ray (see FIGS. 7A and 7B for illustration). FIG. 7A demonstrates a schematic of the region of interest marked by the dispenser 40 and FIG. 7B shows actual appearance of the marking under fluoroscopy. Application of the external medical marking substance on a patient identifies vessels or organs, derived from an initial contrast injection during an imaging procedure (see FIGS. 8A and 8B as examples). A gel-like material is used to create a stationary layer (containing radiopaque contrast media) over selected portions of the skin and/or surgical covers of patients. The stationary layer material is non-toxic, non-allergenic, and readily removable at the completion of the medical procedures. The formula of the material is radiopaque enough to allow the surgeon to visualize markings with the marking substance (and designated dispensing tips on a preferred dispenser) as a line or an area in a real-time manner during the procedures, after initial visualization has been achieved with the first volume of intra-vascular contrast material single injection.

A prospective longitudinal open label cohort study with an internal comparison group (conventional vascular therapeutic interventional procedure patients), and a specific exposure group (external marking device vascular therapeutic interventional procedure patients) was utilized in this study.

### Patient Selection

Patients were randomly selected for the external marking pen evaluation or conventional procedure. These patients had an elective vascular interventional procedure scheduled (arterial or venous) at São Luis Morumbi Hospital Interventional Radiology services, with previous clinical and laboratory evaluations and anesthesiology clearance for the procedures. All patients consented to an interventional vascular therapeutic procedure used non-ionic isosmolar contrast media.

Patients who consented to any vascular therapeutic endovascular procedure were invited to participate in the present study. Patients entering the study were forwarded in randomly in a one-to-one division to the two groups (conventional procedure or external marking device group) regardless of their diagnosis or procedure indications.

Patients subjected to cardiac or neurologic interventional procedures were excluded from the study. There were no specific exclusion criteria for the use of the external marking pen directly on the patient skin or surgical covers.

### Data Collection

Between Aug. 1, 2019 and Nov. 30, 2019, pre- and post-procedure clinical parameters, radiologic imaging findings, contrast volumes, contrast costs, and post-procedure complications were entered into a medical record form, analyzed, and compared. Informed consent from patients for the procedure and publication were obtained. Institutional Board Review permission was granted for analysis and publication of the collected data and images. The demographic data available for patients is shown in Table 2 below.

TABLE 2
Patient Demographics

| | | | | | | | Arterial/ | |
|---|---|---|---|---|---|---|---|---|
| | | % | Mean Age | Age | Male/ | Corresp. | Venous | Corresp. |
| | N | Total | (in years) | Range | Female | Percentage | Procedures | Percentage |
| Total | 316 | 100 | 67.6 | 23-93 | 178/138 | 56.3/43.7 | 249/67 | 78.8/21.2 |
| Diagnostic | 119 | 37.6 | 59.8 | 31-84 | 75/44 | 63.0/37.0 | 100/19 | 84.0/16.0 |
| Therapeutic | 197 | 62.3 | 66.2 | 28-93 | 111/86 | 56.3/43.7 | 149/48 | 75.6/24.4 |
| Therapeutic | 72 | 22.8 | 57.1 | 28-81 | 41/31 | 56.9/43.1 | 50/22 | 69.4/30.6 |
| Vascular | | | | | | | | |
| Procedure | | | | | | | | |
| Conventional | 36 | 11.4 | 58.5 | 34-81 | 21/15 | 58.3/41.7 | 28/8 | 778/22.2 |
| Dispenser | 36 | 11.4 | 55.7 | 28-78 | 17/19 | 47.3/52.7 | 15/21 | 41.6/58.3 |

### Data Analysis

Study data was described using parametric mean summary statistics (means, standard deviations, central tendency measures, ratios and percentages). Data was entered into Excel spreadsheets (Microsoft, Redmond, Wash.—USA) and imported into SPSS v 22 (IBM Corp, Armonk, N.Y.—USA). Continuous variables were compared with an independent samples t-test and to detect significant differences between the two groups and nominal variables were analyzed using Pearson's chi square. Results were considered statistically significant with a p<0.05. All analysis were supervised by a bio statistician.

### Results

The potential for reduction of injected contrast material was assessed in this study reviewing three months (Aug. 1, 2019 to Nov. 30, 2019) of endovascular therapeutic procedures in a Sao Paulo-Brazil hospital and compared to the group of patients treated in the same time period using the external marking pen in adjunction with intravascular contrast media injection to locate and mark the vessel be treated with endovascular interventional techniques.

The total number of procedures between Aug. 1, 2019 and Nov. 30, 2019 was 316. Of this group, 119 patients underwent diagnostic exams (37.6%) and 197 patients had therapeutic endovascular interventional procedures (62.3%), with 72 (22.8% of the therapeutic procedures) vascular cases (Table 1). The 72 consecutive patients submitted to a peripheral vascular therapeutic procedure were divided into two groups in one-to-one fashion: 36 patients treated with conventional endovascular methods and uses of contrast media and 36 patients treated using the external marking device as a substitute for multiple contrast injections. A summary of the 36 patients submitted to conventional vascular therapeutic procedures is described in Table 3 below.

TABLE 3
Summary of conventional vascular
therapeutic procedure patients

| | | | Contrast | |
|---|---|---|---|---|
| Patient | Diagnosis | Procedure | (ml) | Cost |
| TASS | TU | EMB | 185 | $423.65 |
| HHC | CAROT | PROT | 205 | $469.45 |
| EAM | TU | EMB | 215 | $492.35 |
| CACM | HEMORR | EMB | 220 | $503.80 |
| MP | AN | PROT | 230 | $526.70 |
| EJL | EST MES | PLAST | 235 | $538.15 |
| RKRN | IAA | PLAST | 253 | $579.37 |
| GDN | IAA | PLAST | 255 | $583.95 |
| APOF | AN | PROT | 260 | $595.40 |
| HS | EST MES | PLAST | 260 | $595.40 |
| MM | IAA | PROT | 260 | $595.40 |
| CFFS | CAROT | PROT | 260 | $595.40 |
| CEF | AN | PROT | 260 | $595.40 |
| JCAF | AN | PROT | 263 | $602.27 |
| NOS | MAV | EMB | 265 | $606.85 |
| SARC | IAA | PLAST | 270 | $618.30 |
| SAB | AN | PROT | 270 | $618.30 |
| WR | AN | PROT | 270 | $618.30 |
| NNC | AN | PROT | 275 | $629.75 |
| ICSS | CAROT | PROT | 278 | $636.62 |
| RAGT | IAA | TROMBECT | 280 | $641.20 |
| GC | AN | PROT | 280 | $641.20 |
| CMBBF | IAA | PROT | 280 | $641.20 |
| MA | CAROT | PROT | 280 | $641.20 |
| MLGGG | CAROT | PROT | 280 | $641.20 |
| DARM | AN | PROT | 285 | $652.65 |
| ASOB | AN | PROT | 286 | $654.94 |
| NNC | AN | PROT | 290 | $664.10 |
| MANS | HEMORR | EMB | 290 | $664.10 |
| JR | AN | PROT | 290 | $664.10 |
| PAA | AN | PROT | 297 | $680.13 |
| MVS | AN | PROT | 300 | $687.00 |
| LV | AN | PROT | 300 | $687.00 |
| BT | IAA | PROT | 320 | $732.80 |
| JRS | CAROT | PROT | 365 | $835.85 |
| MCH | AN | PROT | 370 | $847.30 |
| Average | | | 271.72 | $622.24 |
| Mean | | | 25.27 | 57.88 |
| Deviation | | | | |
| Standard | | | 36.68 | 84.01 |
| Deviation | | | | |
| Variance | | | 1,345.86 | 7,057.84 |
| Coefficient | | | 13.50 | 13.50 |
| of Variation | | | | |
| (%) | | | | |
| Confidence | | | 11.98 | 27.44 |
| Interval | | | | |
| (95%) | | | | |
| Range | | | 185 | 423.65 |

A summary of the 36 patients submitted to external marking pen (dispenser 40) therapeutic procedures in described in Table 4 below.

TABLE 4
Summary of external marking device vascular
therapeutic procedure patients

| | | | Contrast | |
|---|---|---|---|---|
| Patient | Diagnosis | Procedure | (ml) | Cost |
| RAN | IAA | PLAST | 84 | $192.36 |
| IFP | FAV | PLAST | 86 | $196.94 |
| MAMP | FAV | PLAST | 87 | $199.23 |
| EFN | FAV | TROMBECT | 88 | $201.52 |
| AACC | TU | EMB | 91 | $208.39 |
| RMGC | FVC | PROT | 93 | $212.97 |
| RBS | AN | PROT | 95 | $217.55 |
| LMR | AN | PROT | 95 | $217.55 |
| DNF | IAA | PROT | 96 | $219.84 |
| TOM | MAV | EMB | 96 | $219.84 |
| ALSAB | FAV | PLAST | 98 | $224.42 |
| ATL | TVP MMSS | PLAST | 100 | $229.00 |
| GEQG | IAA | PLAST | 101 | $231.29 |
| EPP | TVP | PROT | 102 | $233.58 |
| HA | IAA | PROT | 103 | $235.87 |
| BYFR | SCP | EMB | 103 | $235.87 |
| MSS | MT | PROT | 105 | $240.45 |
| GCMS | MT | PROT | 105 | $240.45 |
| IFC | EP | TROMBECT | 105 | $240.45 |
| TNF | TU | EMB | 105 | $240.45 |
| GVG | AN | PROT | 105 | $240.45 |
| MDBP | IAA | TROMBECT | 105 | $240.45 |
| FCB | MT | PROT | 105 | $240.45 |
| TNTPG | TVP | TROMBECT | 105 | $240.45 |
| FL | AN | PROT | 105 | $240.45 |
| TK | EP | TROMBECT | 105 | $240.45 |
| AMV | MT | PROT | 107 | $245.03 |
| HS | IAA | PLAST | 107 | $245.03 |
| SJB | SCP | EMB | 108 | $247.32 |
| CEMD | EP | TROMBECT | 109 | $249.61 |
| GLC | SCP | EMB | 110 | $251.90 |
| CTS | SCP | EMB | 110 | $251.90 |
| LFMAR | MT | PROT | 114 | $261.06 |
| GP | MT | PROT | 115 | $263.35 |
| MGS | CAROT | PROT | 115 | $263.35 |
| ESSSB | MT | PROT | 120 | $274.80 |
| Average | | | 102.30 | $234.28 |
| Mean | | | 6.68 | 15.30 |
| Deviation | | | | |
| Standard | | | 8.53 | 19.55 |
| Deviation | | | | |
| Variance | | | 72.90 | 382.31 |
| Coefficient | | | 8.34 | 8.34 |
| of Variation | | | | |
| (%) | | | | |
| Confidence | | | 2.78 | 6.38 |
| Interval | | | | |
| (95%) | | | | |
| Range | | | 36 | $82.44 |

Of the 72 patients, 44 presented with one or more comorbidities (61.1%). The incidence of the three major associated comorbidities in the conventional cases and dispenser 40 cases are presented on Table 5 shown below.

TABLE 5
Patient comorbidities

| | | % | | |
|---|---|---|---|---|
| | N | Total | Comorbidity | % |
| VASCULAR | 72 | 100 | 44 | 61.1 |
| CONVENTIONAL | 36 | 50 | 23 | 63.9 |
| DISPENSER | 36 | 50 | 21 | 58.3 |

The mean amount of contrast material used in the dispenser 40 procedure group was significantly less (102.31 mL±8.54) than the amount used in the conventional procedure group (271.72 mL±36.69; t₍₇₀₎=−26.99, p<0.001). Accordingly, the mean cost for the dispenser 40 procedure group was significantly less ($234.28±$19.55) than the mean cost of the conventional procedure group ($622.24±$84.01; t₍₇₀₎=−26.99, p<0.001), resulting in a total savings of about 62.4%. Further, the range (difference between the highest and the lowest used volumes) of contrast in the two groups were as follows: 185 ml in the conventional treatment group while 36 ml in the external marking device group (i.e., 5.1× greater in conventional group), see Tables 2 and 3. Differences between contrast volumes and costs are listed in Table 6 for all the patients and Table 7 for vascular patients (conventional vs. dispenser 40), both shown below.

TABLE 6
Contrast and Cost Analysis (all patients)

| | | % | Contrast | Mean | | |
|---|---|---|---|---|---|---|
| | N | Total | (ml) | (ml) | Cost | Mean |
| TOTAL | 316 | 100 | 51,426 | 162.74 | $117,765.00 | $372.00 |
| DIAGNOSTIC | 119 | 37.6 | 11,349 | 95.60 | $25,989.00 | $218.00 |
| THERAPEUTIC | 197 | 62.3 | 40,177 | 203.94 | $92,005.00 | $467.00 |

TABLE 7
Contrast and Cost Analysis (Conventional × External Marking Device)

| | | % | Contrast | Mean | | |
|---|---|---|---|---|---|---|
| | N | Total | (ml) | (ml) | Cost | Mean |
| VASCULAR | 72 | 100 | 13,465 | 187.01 | $30,834 | $428.25 |
| CONVENTIONAL | 36 | 50 | 9,782 | 271.72 | $22,400 | $622.22 |
| DISPENSER | 36 | 50 | 3683 | 102.31 | $8,434 | $234.27 |

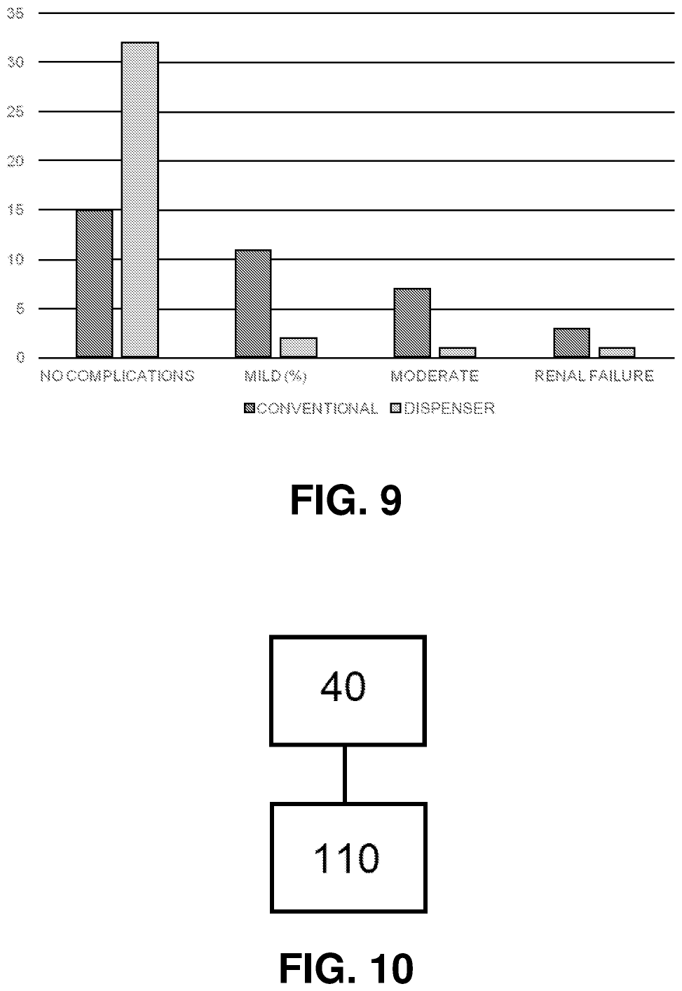

Most importantly, there was a significant relationship between type of procedure (i.e., Conventional or dispenser 40) and Outcome (i.e., No Complications, Mild Complications, Moderate Complications, or Renal Failure), with the dispenser 40 associated with lower incidence of procedure-related complications (c²₍₃₎=17.88, p<0.001; see FIG. 9. The statistical data comparing the two groups are presented in Tables 2 and 3.

Mild complications, defined as local urticaria, nausea or contrast extravasation was observed in 13 of the 72 patients (18.1%). Moderate complications, defined as diffuse urticaria, vomiting, bronchospasm with or without dyspnea or tachycardia or bradycardia was diagnosed in 8 patients (11.1%). Severe complications were defined as renal failure, life-threatening arrhythmias, hypotension, overt bronchospasm, laryngeal edema, syncope or death. Four patients (5.5%) undergoing a vascular procedure developed elevated creatinine levels greater than 1.75 mg/dL (normal baseline: males 0.7 to 1.2 mg/dL and females 0.6 to 1.1 mg/dL), with signs and symptoms indicating acute renal failure. Of these patients, 2 required ICU admission and at least one session of hemodialysis. In this specific group, 1 patient had hypertension and diabetes and the other patient had hypertension. Complications are presented in Table 8 shown below.

TABLE 8
Observed complications (MOD = Moderate; RF = Renal failure)

| | N | % Total | MILD | % | MOD | % | RF | % |
|---|---|---|---|---|---|---|---|---|
| VASCULAR | 72 | 100 | 13 | 18 | 8 | 11.1 | 4 | 5.5 |
| CONVENTIONAL | 36 | 50 | 11 | 30.5 | 7 | 19.4 | 3 | 8.3 |
| DISPENSER | 36 | 50 | 2 | 5.5 | 1 | 2.8 | 1 | 2.8 |

Thirty-six patients were submitted to vascular therapeutic procedures between Aug. 1, 2019 and Nov. 30, 2019 using the external marking pen in addition to intravascular contrast media injection to locate and mark the vessel, to be treated with endovascular interventional techniques. The mean age of these patients was 55.7 years old (28 to 78), being 19 females (52.7%) and 17 males (47.3%). Of the 36 patients, 21 had a diagnosis of systemic comorbidity (58.3%): Hypertension (Hyp) 5 (23.8%) Diabetes (D) 3 (14.2%) Pulmonary Obstructive Disease (POD) 1 (4.7%) Hyp+D 9 (42.8%) D+POD 1 (4.7%) Hyp+D+POD 3 (14.2%).

Mild complications, as defined above (local urticária, náusea or contrast extravasation) was observed in 2 patients (5.5%). Moderate complications (diffuse urticaria, vomiting, brochspasm or arrhythmias) was diagnosed in 1 patient (2.7%). Severe complications were defined as renal failure, life-threatening arrhythmias, hypotension, overt bronchospasm, laryngeal edema, syncope or death. There were no cases of bronchospasm or arrhythmias and no deaths. One patient (2.7%) developed elevated levels of serum creatine (>1.75 mg/dL) but did not require admittance in ICU or hemodialysis. Blood creatinine level was normal in 72 hrs. The patient was a 77 year old male with multi-systemic comorbidities: hypertension and diabetes and congestive heart failure.

### Discussion

As shown above, performing the stuffy revealed that the use of external marking pen (dispenser 40) can substantially reduce the amount of injectable contrast material as well as associated cost in peripheral procedures. Most importantly, the study also revealed significantly lower complication rates in patients who underwent the dispenser 40 procedure due to less contrast material used in the procedures.

These findings suggest that the dispenser 40 may not only be a viable method to be used to visualize vessels, but may be preferable to conventional methods. Since almost all arterial and venous vascular diseases and non-vascular pathologies, such as cancer, uterine fibroids, renal/ureter obstructions, liver fibrosis and others may now be diagnosed and treated with minimally invasive procedures, attributable to advancements in interventional radiology, finding a technique that allows for visualization of vessels with reduced use of contrast material can have significant positive implications for patients in terms of fewer complications and lower costs.

Dispenser 40 may have significant impact in the endovascular field for procedures, especially therapeutic angiography, angioplasty and stenting, treatment of venous and arterial thromboembolic disease, aortic aneurysm repair, inferior vena cava filter placement, vertebroplasty, thoracocentesis and paracentesis, peripheral vascular bypass, carotid angioplasty and stenting, dialysis access, thrombolytic therapy, and others. As demonstrated in this study, the external marking pen significantly reduced the volume of ICM used and reduced the overall procedural cost. These findings may be translated to cardiac, vascular and neurologic endovascular procedures, by reducing the total volume of expensive contrast material. Similar savings may occur through reduced procedure time leading to lower operating room or catheterization laboratory expenses. Patient length of stays in hospitals, particularly those that develop multi-systemic complications related to the use of high volumes of contrast, may also be reduced.

### Study Conclusions

The external marking pen (dispenser 40), when used in conjunction with intravascular contrast media in therapeutic interventional procedures may lower total contrast volumes, thus reducing complications associated with ICM use, especially in patients presenting with multi-systemic comorbidities. Reduction of overall costs by reducing procedure times, avoiding prolonged hospital stays and ICU admissions is another major benefit of the dispenser 40. The external marking pen (dispenser 40) may have significant impact in the endovascular field for procedures including therapeutic angiography, angioplasty and stenting, treatment of venous and arterial thromboembolic disease, aortic aneurysm repair, inferior vena cava filter placement, vertebroplasty, thoracocentesis and paracentesis, peripheral vascular bypass, carotid angioplasty and stenting, dialysis access, thrombolytic therapy and others. Non-vascular disorders are also treated with interventional radiology procedures, using minimally invasive techniques that may also have applicability to the use of the external marking pen in procedures such as neoplasic tumor feeding arteries and vein locations, embolization, immunotherapy, chemoembolization, ablations and other procedures.

While the invention has been described herein with certain parameters and materials, it is understood that the desired parameters are the invention, and may be achieved by use of currently known or equivalent materials. The unique combination of characteristics that is disclosed herein is essential to achieving the goal of effective reduction of exposure of patients to injectable contrast material. While various embodiments of devices and methods of using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. An external medical marking substance for use on a patient during a medical imaging procedure, comprising:
a volume of non-toxic radiopaque material suitable for creating an image on a medical imaging device when said radiopaque material is combined with a forming material and the combination is placed externally on a patient and imaged by medical a imaging device; and
a volume of forming material for combination with the radiopaque material, the forming material providing suitable characteristics for the combined materials forming the marking substance to create both an image visible to the naked eye and a radiographic image when the marking substance is viewed by the medical imaging device.

2. The medical marking substance of claim 1, in which the radiopaque material is selected from the list of radiopaque materials consisting of a mono-acidic monomer, a mono-acidic dimer, a non-ionic monomer, and a non-ionic dimer.

3. The medical marking substance of claim 1, in which the radiopaque material is a volume of inorganic metallic lead particles.

4. The medical marking substance of claim 3, in which the volume of inorganic metallic lead particles comprises particles of more than one shape.

5. The medical marking substance of claim 1, in which the forming material is selected from the list of forming materials consisting of polyglucoside, hydroxyethylcellulose, carboxymethylcellulose, a skin adhesive formed from a polyvinylacetate base, and combinations thereof.

6. The medical marking substance of claim 1, in which the forming material is selected from the list of forming materials consisting of carboxymethyl cellulose and polyglucoside ester.

7. The medical marking substance of claim 1, in which the marking substance is flowable for application onto a patient in a medical imaging procedure at temperatures between 15° C. and 50° C.

8. The medical marking substance of claim 6, in which the medical marking substance exits a dispenser as a gel suspension that rapidly dehydrates and hardens on the surface upon which it is deposited.

9. The medical marking substance of claim 8, in which the hardening time after dispensing is between about 4 seconds and 10 seconds.

10. The medical marking substance of claim 8, in which the viscosity of the marking substance is in a range of about 150,000 cP to about 250,000 cP.

11. The medical marking substance of claim 8, in which the viscosity of the marking substance is in a range of about 150,000 cP to about 250,000 cP within 4 seconds to 10 seconds after exiting the dispenser.

12. (canceled)

13. (canceled)

14. The medical marking substance of claim 1, in which the concentration of the forming material in the medical marking substance is between about 75% and 95%.

15. The medical marking substance of claim 1, in which the concentration of the forming material in the medical marking substance is between about 75% and 85%.

16. The medical marking substance of claim 1, in which the medical marking substance is suitable to create a line-like marking on the patient being imaged that has a thickness of between about 0.5 to about 1.5 mm.

17. The medical marking substance of claim 1, in which the medical marking substance is suitable to create a line-like marking on the patient being imaged that has a width of between about 0.5 to about 1.0 mm.

18. The medical marking substance of claim 1, in which the marking substance is non-toxic externally to the patient and is readily removed with water or a substantially neutral solvent.

19. The medical marking substance of claim 1, configured for use in connection with a dispenser, the dispenser comprising:
a central reservoir section configured to receive the medical marking substance;
a first distal tip located at a first distal end of the dispenser;
a second distal tip located at a second distal end of the dispenser;
a first channel defined within the first distal tip, the first channel arranged in a first orientation; and
a second channel defined within the second distal tip, the second channel arranged in a second orientation different from the first orientation.

20. The medical marking substance of claim 19, whereby the dispenser is configured for use in connection with a medical imaging device to place the medical marking substance externally on the patient and to visibly detect the medical marking substance as it is being dispensed by the dispenser.

21. A dispenser of a medical marking substance, comprising:
a central reservoir section configured to receive a quantity of a medical marking substance therein;
a first distal tip located at a first distal end of the dispenser;
a second distal tip located at a second distal end of the dispenser;
a first channel defined within the first distal tip, the first channel arranged in a first orientation; and
a second channel defined within the second distal tip, the second channel arranged in a second orientation different from the first orientation;
wherein the dispenser is configured to dispense the medical marking material externally on a patient while the patient is undergoing a procedure using a medical imaging device.

22. The dispenser of claim 21, further comprising:
An extension element coupled thereto or formed as part thereof, the extension element configured to facilitate the dispensing of the medical marking material from a distance away from the dispenser so that a user's hand is not detected by the medical imaging device when dispensing the medical marking material.
